(19) United States
(12) Patent Application Publication
(10) Pub. No.: US 2022/0304599 A1
(43) Pub. Date: Sep. 29, 2022

(54) ANALYTE SENSOR

(71) Applicants: Bradley LIANG, Bloomfield Hills, MI (US); Rajiv SHAH, Rancho Palos Verdes, CA (US); PERCUSENSE, INC., Valencia, CA (US)

(72) Inventors: Bradley LIANG, Bloomfield Hills, MI (US); Rajiv SHAH, Rancho Palos Verdes, CA (US)

(21) Appl. No.: 17/638,405

(22) PCT Filed: Aug. 31, 2020
(86) PCT No.: PCT/US2020/048743

§ 371 Date: Feb. 25, 2022

Related U.S. Application Data
(60) Provisional application No. 62/894,781, filed on Aug. 31, 2019.

(51) Int. Cl. A61B 5/1486; G01N 27/327; C12Q 1/00; C12Q 1/32

(52) U.S. Cl. CPC A61B 5/14865; G01N 27/3272; C12Q 1/004; C12Q 1/32

(57) ABSTRACT

In one embodiment, a working electrode measuring the presence of a first analyte is disclosed. The working electrode includes a working conductor that has a first electrode reactive surface. The working electrode further includes a first transport material that enables flux of the first analyte to the first reactive chemistry. Additionally, a first reactive chemistry that is responsive to the first analyte is included in the working electrode. The first reactive chemistry includes a mediator, an enzyme and a cofactor. Wherein the first reactive chemistry is located between the working conductor and the first transport material.

### RELATED APPLICATIONS

This application claims the benefit of U.S. provisional application No. 62/894,781 filed Aug. 31, 2019. The application listed above is hereby incorporated by reference in its entirety for all purposes.

### FIELD OF THE INVENTION

The present invention is generally directed to devices and methods that perform in vivo monitoring of an analyte or analytes such as, but not limited glucose, lactate or ketones. In particular, the devices and methods are for electrochemical sensors that provide information regarding the presence or amount of an analyte or analytes within a subject.

### BACKGROUND OF THE INVENTION

Diabetes is a growing healthcare crisis, affecting nearly 30 million people in the United States. Approximately 10 percent of those affected require intensive glucose and insulin management. In hospital patients, hypoglycemia in both diabetic and non-diabetic patients is associated with increased cost and short- and long-term mortality.

Diabetic ketoacidosis (DKA) is a serious complication of diabetes. Diabetic ketoacidosis most often occurs in those with type 1 diabetes though it can also occur those with other types of diabetes. DKA typically occurs when high levels of blood acids called ketones are produced. The condition develops is associated with diabetes because it is linked to the lack of insulin production. Without enough insulin, the body switches to burning fatty acids, which results in production of acidic ketone bodies.

Accordingly, it would be highly advantageous to enable real-time in-vivo detection and measurement of ketone bodies. The claimed invention seeks to address many issues associated with detecting and measuring ketone bodies.

### BRIEF SUMMARY OF THE INVENTION

In one embodiment, a working electrode measuring the presence of a first analyte is disclosed. The working electrode includes a working conductor that has a first electrode reactive surface. The working electrode further includes a first transport material that enables flux of the first analyte to the first reactive chemistry. Additionally, a first reactive chemistry that is responsive to the first analyte is included in the working electrode. The first reactive chemistry includes a mediator, an enzyme and a cofactor. Wherein the first reactive chemistry is located between the working conductor and the first transport material.

In another embodiment, an electrochemical sensor for measuring in-vivo analyte concentration within a subject is disclosed. The electrochemical sensor has a working electrode that includes a working conductor with an electrode reactive surface. The working electrode further includes a reactive chemistry that is responsive to an analyte. Additionally, the reactive chemistry is applied over the electrode reactive surface and also includes a mediator, an enzyme and a cofactor. The electrochemical sensor further includes a pseudo-reference electrode having a combined counter-reference conductor. Where a transport material applied over the reactive chemistry and the pseudo-reference electrode enables flux of the analyte to the reactive chemistry.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1 is an exemplary illustration of a cross-section of an electrode, in accordance with embodiments of the present invention.

FIG. 2A is an exemplary cross-section illustration of an alternative embodiment of the electrode, in accordance with an embodiment of the present invention.

FIG. 2B is an exemplary illustration of a cross-section of an electrode, in accordance with embodiments of the present invention.

FIGS. 3A-3D are exemplary illustrations of formation of the reactive chemistry on the electrode reactive surface, in accordance with embodiments of the present invention.

FIG. 4A is an exemplary illustration of electrochemical reactions occurring in proximity of an electrode with an applied potential, in accordance with embodiments of the present invention.

FIG. 4B is an exemplary illustration of the response of a sensor utilizing the techniques described in FIG. 4A, in accordance with an embodiment of the present invention.

FIG. 4A-1 is an exemplary illustration of an alternative embodiment of electrochemical reactions occurring in proximity of an electrode with an applied potential, in accordance with embodiments of the present invention.

FIG. 4B-1 is an exemplary illustration of the response of a sensor utilizing the techniques described in FIG. 4A-1, in accordance with an embodiment of the present invention.

FIG. 4C is a visual representation of the results of cyclic voltammetry during formation of the reactive chemistry by the electropolymerization of the mediator in the presence of the electrode reactive surface, the enzyme and the cofactor, in accordance with embodiments of the present invention.

FIGS. 5A-5C are non-limiting exemplary illustrations of different sensor assemblies that utilize the electrodes discussed above, in accordance with embodiments of the present invention.

### DETAILED DESCRIPTION

Presented below are embodiments of an electrode within a sensor that is intended to enable continuous real-time in-vivo electrochemical sensing of an analyte within a subject. The in-vivo measurement within a subject is typically performed in tissue such as, but not limited to subcutaneous tissue. However, various embodiments can be inserted into the vasculature, musculature or organ tissue. The sensor may include a working electrode along with a counter electrode and a reference electrode. Alternatively, many embodiments utilize a working electrode in conjunction with a pseudo-reference electrode (combined counter-reference electrode).

Embodiments of the sensor can be configured to measure analytes such as lactate, ketones, glucose and the like. Furthermore, while some embodiments may be configured to measure a single analyte, other embodiments can be configured to measure multiple analytes such as, but not limited to combinations of lactate, ketone, glucose, oxygen, reactive oxygen and the like. In still other embodiments, the sensors may be configured with infusion sets to enable sensing and delivery of an infusate from a single point of entry.

In many embodiments electrochemical detection of the desired analyte is accomplished using an enzymatic reaction. In many embodiments presented below, the enzymes are selected from the dehydrogenase family. Non-limiting, exemplary dehydrogenase enzymes include glucose dehydrogenase and 3-hydroxybutyrate dehydrogenase. In other embodiments, it may be possible to use alternate enzymes such as, oxidases like glucose oxidase lactate oxidase. In many embodiments the enzyme is immobilized or trapped within a reactive chemistry. In some embodiments, the reactive chemistry includes a mediator and cofactor, alternatively referred to as a coenzyme. The inclusion of the cofactor can improve the ability to measure analytes having generally low endogenous concentrations of cofactor. Particularly, inclusion of the cofactor can improve linearity of the sensor response to increasing concentrations of the analyte. Furthermore, inclusion of the mediator can improve performance of the electrochemical sensor by reducing the overpotential required to oxidize reactants. This low applied potential also eliminates possible causes of interference from other electroactive molecules that may exist in the surrounding environment.

The various embodiments discussed below should not be viewed as discrete embodiments. Rather, it is intended that various elements or components of the various embodiments are intended to be combined with elements, features or components of the other embodiments. While embodiments and examples may be related to particular figures the scope of the disclosure and claims should not be construed to be limited to the explicit embodiments discussed. Rather it should be recognized that various combinations of features, elements and components can be interchanged, combined and even subtracted to enable other embodiments capable of continuous, real-time detection and measurement of an analyte or multiple analytes indicative of various metabolic conditions or general physiological health.

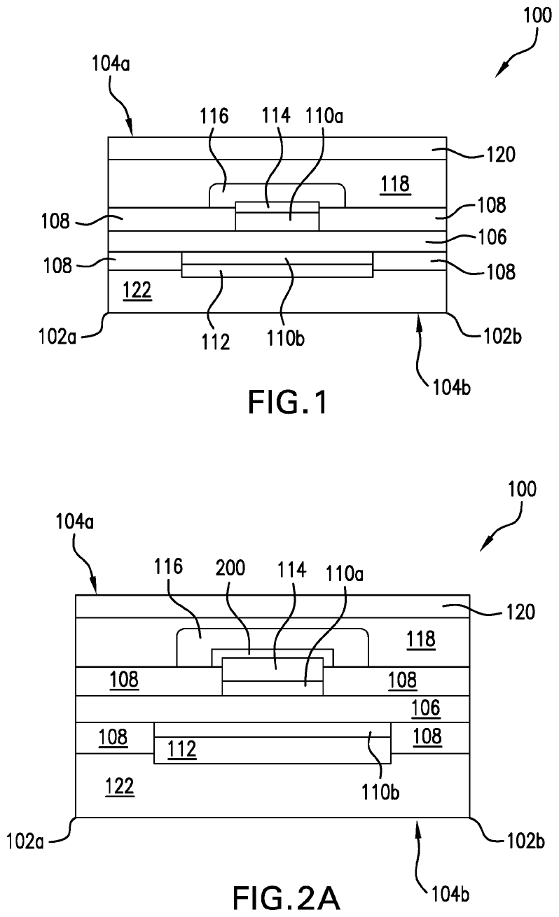

FIG. 1 is an exemplary illustration of a cross-section of an electrode 100, in accordance with embodiments of the present invention. The cross-section illustrated in FIG. 1 includes edges 102a and 102b along with top 104a and bottom 104b. Insulation 106 is located between working conductor 110a and counter/reference conductor 110b. In some embodiments, the working conductor 110a and the counter/reference conductor 110b are made of the same material. In other embodiments, the working conductor 110a and the counter/reference conductor 110b are made from different materials. The rationale for selecting different materials for the working conductor and the counter reference conductor include, but are not limited to corrosion resistance, electrical conductivity, material properties that enable electroplating, In many embodiments both the working conductor 110a and the counter/reference conductor 110b are selected from conductive materials such as, but not limited to stainless steel, silver, platinum, copper and the like. Preferable material qualities for both the working conductor 110a and the counter/reference 110b include flexibility, ductility and toughness.

While the embodiment shown in FIG. 1 has a single conductor that is used as a combined counter/reference, other embodiments may use individual separate conductors resulting in a counter conductor and a reference conductor being formed on separate and discrete conductors. In many embodiments the working conductor 110a further includes an electrode reactive surface 114. Similarly, the counter/reference conductor 110b may further include a surface treatment 112. In some embodiments both the electrode reactive surface 114 and the surface treatment 112 are optionally applied. In still other embodiments either the electrode reactive surface 114 or the surface treatment 112 may be optionally applied.

In many embodiments an electroplating process is used to create the electrode reactive surface 114 and the surface treatment 112. Exemplary electroplating materials include, but are not limited to gold, silver, platinum and the like. Insulation 106 separates and electrically isolates working conductor 110a and counter/reference conductor 110b. Furthermore, the working conductor 110a and the counter/reference conductor 110b are distanced from edges 102a and 102b by insulation 108. In other embodiments, the electrode reactive surface 114 may be disposed upon the working conductor 110a using alternative processes such as, but not limited to printing, sputtering, chemical vapor depositing and the like. In many embodiments conductive pastes, gels or inks may be used that include non-limiting, exemplary electrically conductive elements or compounds such as carbon, graphite, graphene, silver, silver-chloride, platinum or mixture combinations thereof.

As illustrated in FIG. 1, a reactive chemistry 116 is applied over the electrode reactive surface 114. In embodiments that do not utilize an electrode reactive surface, the reactive chemistry 116 may be optionally applied directly to the working conductor 110a. In the embodiments illustrated in FIG. 1, the reactive chemistry 116 may be mixtures or compounds that include immobilized enzyme. The particular enzyme immobilized within the reactive chemistry 116 may be selected based on its ability to react with the analyte the electrode is configured to measure. Other materials within the reactive chemistry 116 include, but are not limited to hydrogels and other polymerizing agents selected based on their ability to immobilize the enzyme within the reactive chemistry 116. Exemplary enzymes within the reactive chemistry 116 include, but are not limited to oxidases (e.g., glucose oxidase, lactate oxidase, and the like) along with dehydrogenases (e.g., glucose dehydrogenase, 3-hydroxybutyrate dehydrogenase and the like).

A first transport material 118 is applied over the reactive chemistry 116. The first transport material 118 is typically selected from a family of three-dimensional hydrogels that enable omnidirectional transport of tissue fluid that surrounds the sensor after insertion into a subject. Applied over the first transport materials 118 is a second transport materials 120. In many embodiments the second transport material 120 is considered optional. The second transport material 120 may be selected based on a variety of factors such as, but not limited to its ability to physically protect the underlying structure, ability to transmit or attenuate desired compounds or reactants, and how impermeable the second transport materials 120 is to analytes/reactants within the surrounding tissue.

A third transport material 122 is applied over the surface treatment 112 or alternatively, the counter/reference electrode 110b. In many embodiments, the third transport material 122 is identical to the second transport material 120. Similarly, the selection of the third transport material 122 may be based on similar characteristics of physical toughness, transmission, attenuation and impermeability. In some embodiments it may be desirable for the third transport material 122 to have different characteristics than the second transport material 120. The first, second and third transport materials are typically hydrogel or doped hydrogels. However, in various embodiments any of the transport materials could be other permeable, semi-permeable, or non-permeable materials such as, but not limited to a crosslinked albumin membrane, polyethylene glycol) diacrylate (PEGDA), polyurethane, silicone and the like. The optional second transport material 120 may be used when it may be desirable to have a longer diffusion pathway between the implant environment at the edges 102a and 102b and the reactive chemistry 116.

Furthermore, additional transport materials or blends/mixtures of transport materials can be used to entrap or enable transport of supporting molecules. In various embodiments, transport materials may be disposed upon the sensor in a pattern best suited to enable transport of desirable molecules or, alternatively, reject or impede transport of undesirable molecules. Non-limiting exemplary application patterns for transport materials include application of the transport materials discretely over the reactive chemistry or blanketing the entire top 104a of the sensor. Another application pattern for transport materials includes blanketing the counter/reference electrode 110b or surface treatment 112.

The embodiment illustrated in FIG. 1 is intended to be exemplary and non-limiting. In particular, the relative dimensions should not be construed as limiting or exemplary of actual relative dimensions of various components within the sensor. For example, the relative thickness of the third transport material 122 to the second transport material 120, or even the thickness of insulation 106 to the thickness of insulation 108 should not be construed from FIG. 1. Moreover, the relative placement and location of elements should not be construed as limiting. For example, in FIG. 1 the reactive chemistry 116 is depicted as being applied in a larger, substantially concentric nature that overlaps or overshadows the working conductor 110a. This particular illustration should not be construed as limiting. In other embodiments the reactive chemistry 116 can be applied to be substantially concentric while being substantially equal or smaller in size and/or shape than the working conductor 110a. In still other embodiments the reactive chemistry need not be applied over the working conductor in a substantially concentric nature. Rather, in many embodiments the reactive chemistry 116 can be applied as a blanket from edge 102a to edge 102b. FIG. 1 is intended to be an exemplary illustration of the relative placement of components rather than relative size of the respective components.

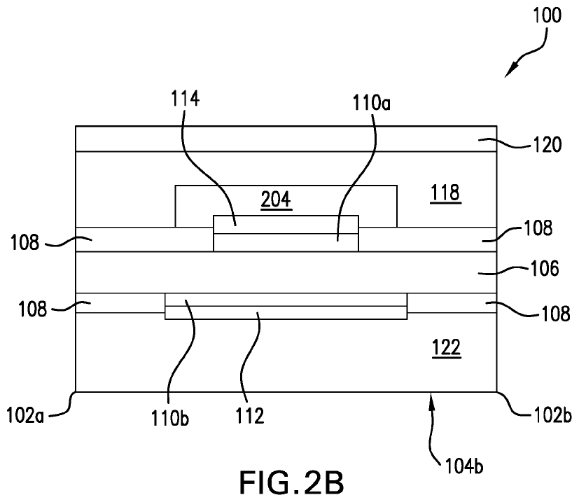

FIG. 2A is an exemplary cross-section illustration of an alternative embodiment of the electrode 100, in accordance with an embodiment of the present invention. The embodiment illustrated in FIG. 2A differs from the embodiments in FIG. 1 in that a mediator 200 is applied between the reactive chemistry 200 and the electrode reactive surface 114. Recall that in FIG. 1, the active chemistry 116 applied directly over the electrode reactive surface 114. In embodiments having the mediator 200, the mediator 200 is selected based on its ability to serve as an electron donor/acceptor. Exemplary mediators 200 include, but are not limited to, organic compounds such as phenothiazine derivatives (e.g. thionine) and phenanthroline derivatives.

In many embodiments the mediator 200 is electropolymerized onto the electrode reactive surface 114. Exemplary, non-limiting electropolyerization protocols for the mediator 200 include cyclic voltammetry using a low frequency triangle wave. Another electropolymerization protocol uses a constant potential signal (chronoamperometry). Another electropolymerization protocol uses a custom signal that incorporates both high frequency and high amplitude. Still another electropolymerization protocol uses multi-step voltammetry. An exemplary multi-step voltammetry protocol polarizes the electrode reactive surface 114 in the presence of the mediator in both positive and negative directions, further including a bias toward positive either in amplitude or duty cycle. Additionally, the exemplary multi-step voltammetry protocol is conducted at a constant voltage.

The electropolymerization techniques discussed above are exemplary and should not be considered restrictive. Different electropolymerization techniques can be utilized to modify or tune the properties of the mediator 200. In the configuration illustrated in FIG. 2A, the presence of the mediator 200 enables the electrode to operate at a lower electrical potential in order to oxidize a cofactor, alternatively referred to as a coenzyme, that is associated with the analyte being measured via the reactive chemistry. One benefit of the electrode design illustrated in FIG. 2A is a reduction of interference from electroactive materials or compounds such as peroxide, acetaminophen and the like, relative to the configuration shown in FIG. 1.

With the mediator 200 electropolymerized over the electrode reactive surface 114, the reactive chemistry 116 is applied over the mediator 200. Subsequently, the first transport material 118 envelopes or covers the reactive chemistry 202. As in FIG. 1, a second transport material 120 can optionally be applied over the first transport material 118. Also, as illustrated and as discussed regarding FIG. 1, a third transport material 122 may be applied over the counter/reference electrode 110b and the surface treatment 112.

FIG. 2B is an exemplary illustration of a cross-section of an electrode 100, in accordance with embodiments of the present invention. The embodiment illustrated in FIG. 2B differs from the embodiments in FIG. 1 and FIG. 2A is that reactive chemistry 204, applied between the electrode reactive surface 114 and the first transport materials 118, includes a mediator, an enzyme and a cofactor. In reactive chemistry 204 the enzyme can be selected from a family of dehydrogenase enzymes. The cofactor within the reactive chemistry 204 may be selected based on its ability to effectively catalyze the chemical reaction of the dehydrogenase enzyme. In other words, one criteria when selecting a cofactor may include how well the cofactor functions as an electron acceptor/donor. In many embodiments the dehydrogenase enzyme may be selected from a family where the enzyme is reversible. Non-limiting exemplary reversible dehydrogenase enzymes include lactate dehydrogenase and 3-hydroxybutyrate dehydrogenase.

In an embodiment where the reactive chemistry 204 utilizes 3-hydroxybutyrate dehydrogenase as the enzyme and NAD+ as the cofactor, 3-hydroxybutyrate dehydrogenase can operate with NAD+ in close proximity to effectively oxidize 3-hydroxybutyrate to acetoacetate as the primary reaction byproduct. Reversing the reaction, acetoacetate can be reduced to 3-hydroxybutyrate. The use of NAD+ as the cofactor should not be construed as limiting. Other exemplary cofactors, or electron acceptors/donors, include but are not limited to nicotinamide adenine dinucleotide phosphate (NADP+) and flavin adenine dinucleotide (FAD).

The selection of the mediator within the reactive chemistry 204 may be made using similar criteria to select the mediator for the embodiment shown in FIG. 2A. Specifically, some desirable characteristics of the mediator within the reactive chemistry 204 include a conductive polymer capable of being deposited using an electropolymerization reaction. In various embodiments, it may be beneficial to use an organic azine mediator such as, but not limited to toluidine blue, thionine, 1,10-phenanthroline-5,6-drone, meldola's blue, and methylene blue. Note that using an azine mediator is fundamentally different than using a mediator selected from the family of transition metals. One fundamental difference that distinguishes azine mediators from transition metal mediators is that some azine mediators are better suited for anodes while other azine mediators are better suited for cathodes. Furthermore, the selection or properties of the electrode reactive surface 114, and conditions of electropolymerization ultimately dictate conductance of the resulting mediator-enzyme-cofactor matrix, where the mediator can influence the performance of electron transfer or electron acceptance. Differences in azines may be leveraged depending on their relative affinity to be anodic or cathodic. However, this does not mean an azine must behave as an insulator in one direction and a conductor in the opposite direction. Rather, azine performance as an anode or cathode can be viewed as a spectrum rather than a binary property.

Alternatively, the mediator within the reactive chemistry 204 may be selected from phenothiazine- and phenanthroline-based materials or derivatives thereof, such as those discussed regarding FIG. 2A. These materials have the benefit of operating efficiently as either an anode or cathode with regards to signal amplitude and/or stability.

In an alternate embodiment additional enzyme material can be applied over the reactive chemistry 204, thereby creating an embodiment that is similar, yet slightly different, to the illustration in FIG. 2A. In this alternate embodiment, the reactive chemistry 116 is applied over the reactive chemistry 204. The application of supplemental reactive chemistry 116 over the reactive chemistry 204 can help increase sensitivity of the sensor. Additionally, the supplemental reactive chemistry 116 over the reactive chemistry 204 can further improve sensor stability and be utilized to adjust, or tune, redox reactions equilibriums. The remainder of the structure of the electrode 100 in FIG. 2B is similar to those discussed in FIGS. 1 and 2A.

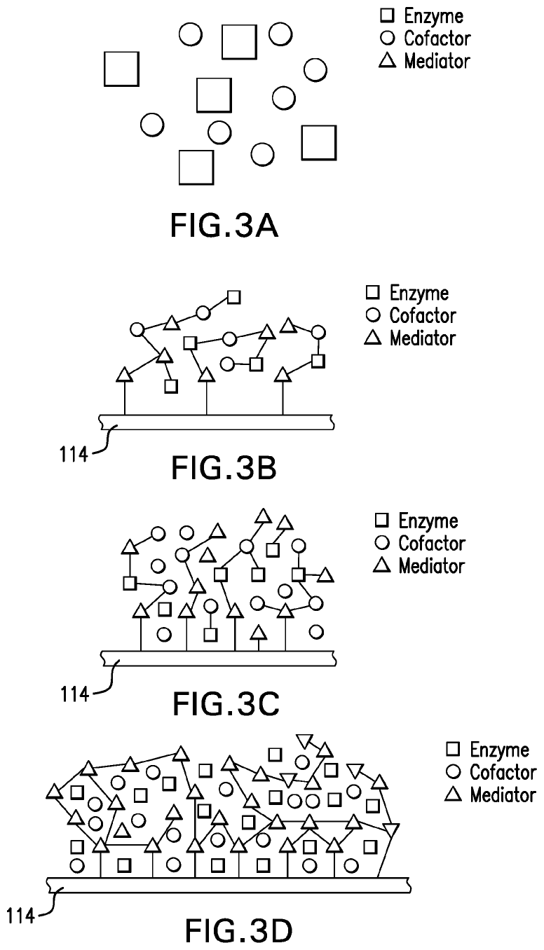

FIGS. 3A-3D are exemplary illustrations of formation of the reactive chemistry 204 on the electrode reactive surface 114, in accordance with embodiments of the present invention. FIG. 3A is an exemplary illustration of a solution containing elevated concentrations of enzyme 300 and cofactor 302. FIGS. 3B and 3C are exemplary illustrations of an electropolymerization of the enzyme 300 and cofactor 302 in the presence of a mediator 304 and the electrode reactive surface 114. The non-limiting exemplary polymerized structure, or matrix, in FIGS. 3B, 3C and 3D contain a mixture of various permutations of the mediator 304, the enzyme 300 and the cofactor 302. In various embodiments, covalent bonds and/or electrostatic bonds and/or combinations thereof are formed between the various combinations of mediator 304, enzyme 300 and cofactor 302.

After electropolymerization, the structure, referred to as reactive chemistry 204, is a mixture of covalent and electrostatic bonds where the mediator 304 can be terminated by mediator 304, enzyme 300, cofactor 302 or combinations thereof. The reactive chemistry 204 may include wired elements of enzyme 300 and cofactor 302 which means the enzyme 300 and/or cofactor 302 are in direct electrical connection with the electrode reactive surface 114 resulting in the mediator 204 participating in the transfer of electrons. The reactive chemistry 204 also includes free elements of enzyme 300 and cofactor 302. Free elements within the reactive chemistry 204 can generally be understood to be a polymer that is immobilized but not in direct electrical connection with the electrode reactive surface 114. Note that in FIG. 3D, the matrix includes only enzyme and cofactor molecules that are free elements in that they are not electrically wired to the electrode reactive surface 114. The mixture of wired elements and free elements enables reversible enzymes, such as, but not limited to dehydrogenase based enzymes, to be utilized and creates a framework that enables the electrode to respond to concentrations of analyte in their presence.

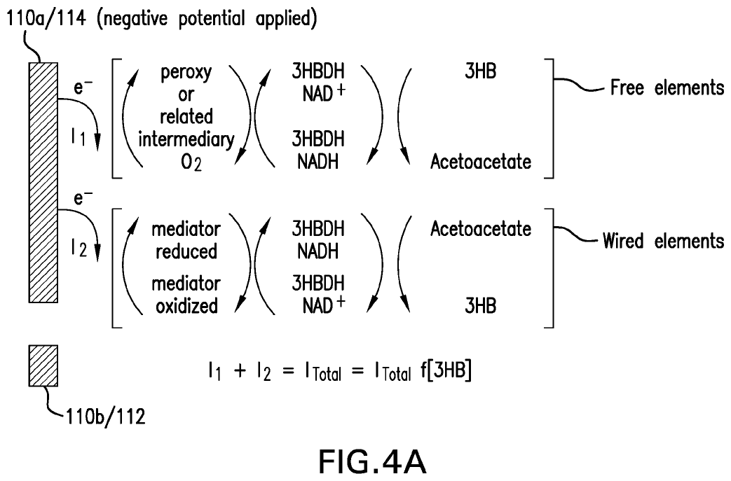

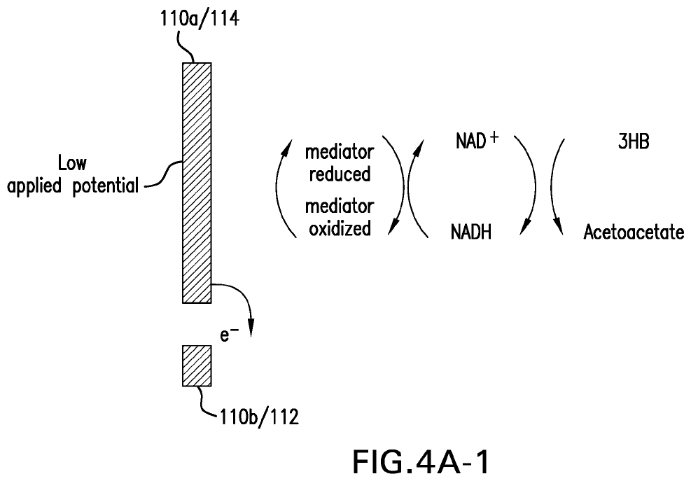

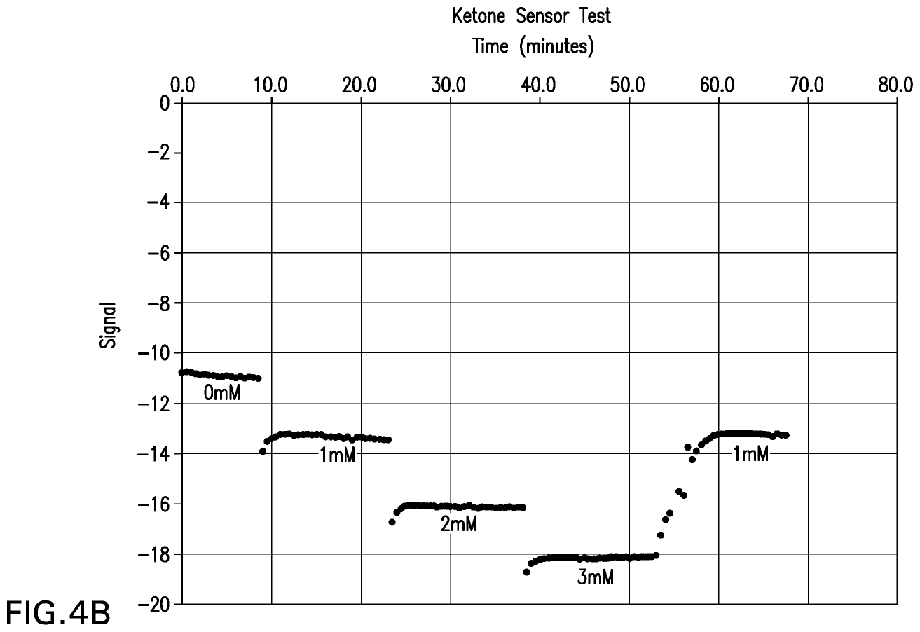

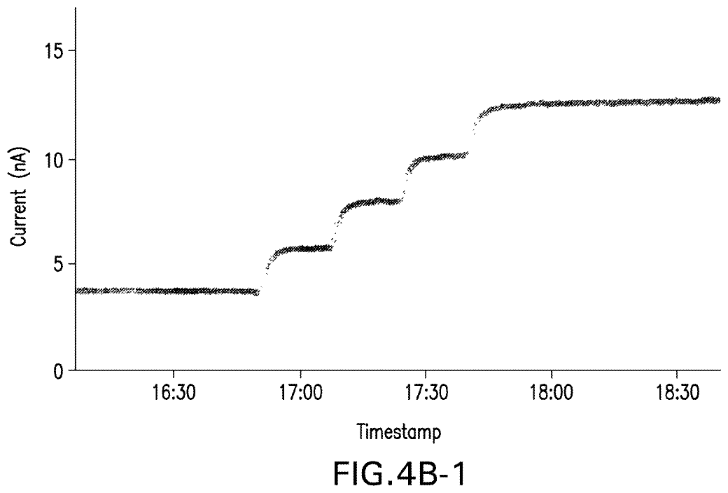

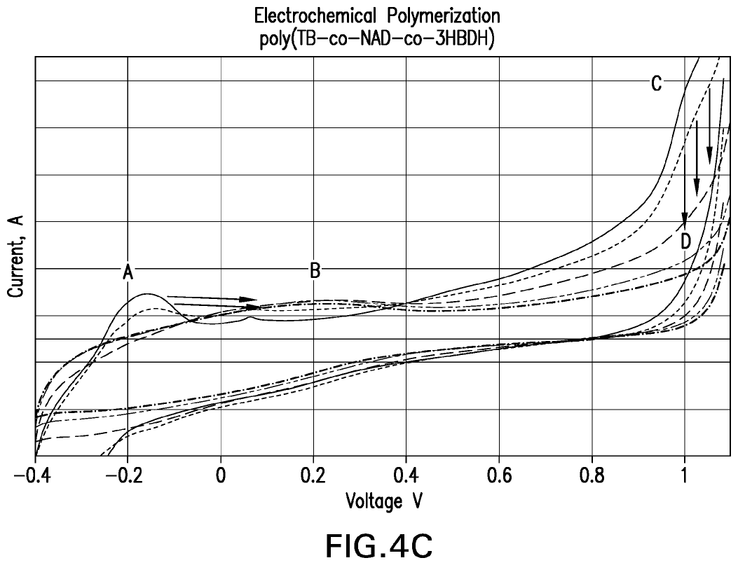

FIG. 4A is an exemplary illustration of electrochemical reactions occurring in proximity of an electrode with an applied potential, in accordance with embodiments of the present invention. In the embodiment illustrated in FIG. 4A an exemplary, non-limiting phenothiazine-derivative mediator such as toluidine blue is polymerized in close proximity with an enzyme and cofactor. In this particular non-limiting example, the enzyme dehydrogenase such as 3-hydroxybutyrate dehydrogenase (3HBDH) and the cofactor is NAD+. Furthermore, the analyte being detected or measured is 3-hydroxybutyrate (3HB), also commonly referred to as ketone.

As illustrated in FIG. 4A, the working electrode 110a is operating as a cathode and accordingly has a negative potential applied. Free element 3HBDH reacts with 3HB to form acetoacetate. Similarly, free element NAD+ forms NADH while acetoacetate generated from the free elements diffuses to wired elements and results in cathodic electron transfer along the wired elements. The direct electron transfer along the wired elements from the cathode to the NAD+ converts wired element NAD+ to NADH. Additionally, NADH supports the reversible action of acetoacetate back to 3HB. Thus, as concentrations of ketone (RIB) goes up, acetoacetate increases thereby driving the electrical current negative. The wired and free element structure of the electropolyerized matrix enables generation of a distinct cathodic signal as electrons are dumped to the electropolymer and delivered to NAD+ that becomes NADH. In one embodiment the working electrode is operated at a negative potential to support a cathodic reaction. The result of operating the working electrode as a cathodic reaction is a reduction, or decrease, in current measured between the electrode reactive surface and the counter/reference electrode, or pseudo-reference electrode as the concentration, or presence of analyte increases. In other embodiments, the working electrode supports an anodic reaction resulting in an increase in current measured between the electrode reactive surface and the counter/reference electrode.

With the reactions illustrated in FIG. 4A, oxidizing agents are reduced but are very active and at low applied potentials, it may be possible for oxidizing agents to build up because they are not fully reacting. Oxidizing agents at the electrode reactive surface may cause free element NADH to go back to NAD+. Furthermore, under an applied potential, free element NADH may be electrochemically oxidized back to NAD+. The embodiment shown in FIG. 4A and discussed above should not be construed as limiting. Various other embodiments can utilize different enzymes and cofactors. Similarly alternative mediators may be used to entrap the enzyme and cofactor within the electropolymerized layer.

FIG. 4B is an exemplary illustration of the response of a sensor utilizing the techniques described in FIG. 4A, in accordance with an embodiment of the present invention. As shown in FIG. 4B, as the concentration of the analyte being measured, ketone (3HB), increases from zero millimolar to three millimolar, the signal generated by the electrode decreases. FIG. 4A further illustrates the reversibility of the reaction because removal and replacement of two-thirds of the fluid with zero millimolar concentration buffer results in the signal returning to the previously measured value.

FIG. 4A-1 is an exemplary illustration of an alternative embodiment of electrochemical reactions occurring in proximity of an electrode with an applied potential, in accordance with embodiments of the present invention. As illustrated in FIG. 4A-1, free elements of 3HBDH oxidize 3HB in the presence of coenzyme NAD+, generating free acetoacetate and NADH. The generated NADH is in turn electrochemically oxidized at the polymerized mediator surface. The oxidation of NADH generates free electrons that may be measured as electrical current and returns the coenzyme to its electron-acceptor state (NAD+). The entrapment of free NAD+ within the polymerized layer ensures an abundance of coenzyme available to the 3HBDH, allowing the generated sensor current to increase linearly with increasing concentrations of 3HB. The mediator allows for oxidation of NADH to occur at low applied potentials, reducing the possibility of interference from other electroactive species such as peroxide, acetaminophen and the like.

FIG. 4B-1 is an exemplary illustration of the response of a sensor utilizing the techniques described in FIG. 4A-1, in accordance with an embodiment of the present invention. FIG. 4B-1 shows that as the concentration of the analyte being measured, ketone (3HB), increases from 1 millimolar to 5 millimolar, the signal generated by the electrode increases.

FIG. 4C is a visual representation of the results of cyclic voltammetry during formation of the reactive chemistry by the electropolymerization of the mediator in the presence of the electrode reactive surface, the enzyme and the cofactor, in accordance with embodiments of the present invention. Specifically, FIG. 4C illustrates cyclic voltammetry curves from a single polymerization run (cycle #5-n), depicting the change in the current as the polymer layer is grown over time. In area A, initially a narrow couple of the mediator is observed in the region of mediator rejection (monomer). However, as higher voltages are achieved during an anodic sweep, area C, the generation of free radicals allows for the formation of a wide couple mediator dimer/oligomer, observable at higher potential range versus than the monomer (area B).

As the cycle count increases, the polymer layer continues to grow. The layer growth corresponds to a reduction in conductivity of the layer, resulting in a reduction in the rate of radical generation (area [D]). Over an increasing number of cycles, the polymerization reaction slows and eventually halts as the conductivity of the electrode is reduced. The growth of the polymer can be affected depending on the pH, temperature, and concentration of species within the polymerization solution. Polymerization processes may be more rapid at lower pH and a preferred solution pH level can be determined based on the particular enzyme and cofactor being used. As discussed in FIG. 4A, in some embodiments electrochemical polymerization is performed using 3HBDH as the enzyme and NAD+ as the cofactor. In many embodiments it may be preferable to choose a weak acid/neutral buffer solution to preserve enzyme activity.

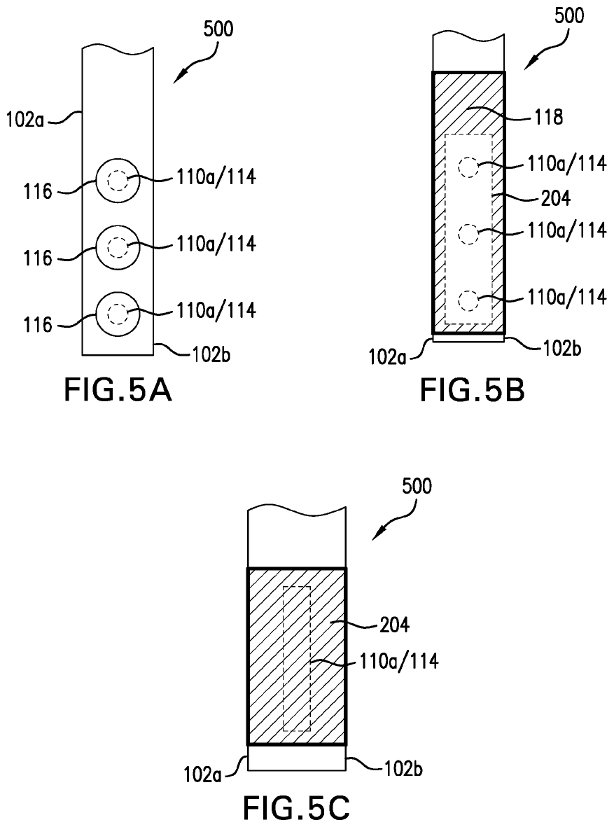

FIGS. 5A-5C are non-limiting exemplary illustrations of different sensor assemblies that utilize the electrodes discussed above, in accordance with embodiments of the present invention. The illustrations in FIGS. 5A-5C should not be construed as including the entirety of a sensor. Rather, in FIGS. 5A-5C, the portion of the sensor containing the working electrodes is presented to provide exemplary illustrations of various configurations for the electrodes described above. Features such as, but not limited to electrical contact pads are intentionally omitted to simplify the figures. FIG. 5A is an exemplary illustration of a sensor assembly 500 that has multiple electrodes such as those illustrated in FIG. 1. For simplicity FIG. 5A shows the placement of exposed working conductor 110a and electrode reactive surface 114 relative to the reactive chemistry 116. In this embodiment, the reactive chemistry 116 is applied in discrete locations substantially concentric with the working conductor 110a and the electrode reactive surface 114. The illustration of the reactive chemistry 116 being applied in a larger, substantially concentric nature that overlaps or overshadows the working conductor 110a should not be construed as limiting. In other embodiments the reactive chemistry 116 could be applied to be substantially concentric while being substantially equal or smaller in size and/or shape than the working conductor 110a. Also note the relative location of both the working conductor 110a, the electrode reactive surface 114 and the reactive chemistry 116 relative to the edges 102a and 102b.

While the working conductor 110a, the electrode reactive surface 114 and the reactive chemistry 116 are shown as being substantially circular, the graphical representation of the elements should not be construed as limiting. In other embodiments various shapes, including different shapes for each element may be used. Likewise while shown being substantially centered between the edges 102a and 102b, other embodiments can have the exposed working conductor 110a, electrode reactive surface 114 and reactive chemistry 116 biased toward either edge 102a or edge 102b. For simplicity, the first transport materials, the second transport material and the elements on an opposite side of the electrode 100 are not shown in FIG. 5A.

FIG. 5B is an exemplary illustration of a sensor assembly 500 that has multiple electrodes such as those illustrated in FIG. 2B. The working conductor 110a and electrode reactive surface 114 are shown as circles. In this embodiment, the reactive chemistry 204 is applied as the dashed rectangle that continuously covers the working conductor 110a and electrode reactive surface 114. The embodiment shown in FIG. 5B can be differentiated from FIG. 5A in that the reactive chemistry 204 is applied more as a blanket coating over all of the working electrodes 110a rather than discreetly over each respective working electrode. This embodiment can ensure an abundance of reactant and cofactor to react with the analyte being measured. FIG. 5B further includes an illustration of first transport materials 118 blanketing the entirety of the reactive chemistry 116 and the working electrodes 110a and electrode reactive surface 114.

FIG. 5C is still another exemplary illustration of a sensor assembly 500 having a single working electrode 110a. Though the working electrode 110a in FIG. 5C is illustrated as a rectangular slot, the shape of the working electrode should not be construed as limiting. In other embodiments the working electrode can include other shapes such as circles, ellipses, rectangles, and other polygons. Furthermore, the position of the working electrode should not be construed as limiting. In other embodiments, the working electrode 110a can be shifted, or biased toward either edge 102a or edge 102b. In this embodiment, the reactive chemistry 204 blankets the working electrode 110a and further extends the entire width of the sensor from edge 102a to edge 102b.

The embodiments illustrated in FIGS. 5A-5C are intended to be exemplary and should not be construed as limiting. Various other embodiments can incorporate features, designs and elements such as those discussed in U.S. patent application Ser. No. 15/472,194, filed Mar. 28, 2017 and Patent Cooperation Treaty application serial number PCT/US18/38984 filed Jun. 22, 2018, all of where are herein incorporated by reference for all purposes. Furthermore, while the above description discusses the operation of a sensor measuring a single analyte, the electrodes described above can be incorporated with or without modifications into sensors capable of simultaneously measuring multiple analytes.

The embodiments discussed above are typically illustrated using a combined counter electrode and reference electrode, commonly referred to as a pseudo-reference electrode. It should be noted that the working electrode structure disclosed is also capable of operation with a separate counter electrode and reference electrode. In many embodiments, the pseudo-reference electrode is located on a side opposite the working electrode. This dual sided configuration can help reduce the physical width of the sensor, albeit with a marginal increase in the depth or thickness of the sensor. A further benefit of the dual sided configuration is the ability to apply the third transport material over the counter/reference electrode. In single sided embodiments it may be more difficult to tune sensor performance using different transport materials because of limitations on placement of the various transport materials relative to each other. Application of the third transport material is greatly simplified with the counter/reference electrode being on a side completely opposite the working electrode.

In many embodiments, additional features or elements can be included or added to the exemplary features described above. Alternatively, in other embodiments, fewer features or elements can be included or removed from the exemplary features described above. In still other embodiments, where possible, combination of elements or features discussed or disclosed incongruously may be combined together in a single embodiment rather than discreetly as in the exemplary discussion.

Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. A working electrode measuring the presence of a first analyte, comprising:
a working conductor having a first electrode reactive surface;
a first transport material that enables flux of the first analyte to the first reactive chemistry; and
a first reactive chemistry being responsive to the first analyte, the first reactive chemistry including a mediator, an enzyme and a cofactor,
wherein the first reactive chemistry is located between the working conductor and the first transport material.

2. The working electrode described in claim 1, wherein the mediator is a conductive polymer.

3. The working electrode described in claim 2, wherein the first reactive chemistry is formed by electropolymerization of the mediator in the presence of the enzyme and the cofactor.

4. The working electrode described in claim 3, wherein the first reactive chemistry is defined by free elements of the enzyme and the cofactor entrapped within the polymerized mediator.

5. The working electrode described in claim 4, wherein the first reactive chemistry is further defined by wired elements of enzyme and cofactor that are electrically connected to the working electrode and the mediator participates in transferring electrons.

6. The working electrode described in claim 5, wherein reaction of the analyte with free enzyme generates an intermediary and reaction of the free enzyme with the cofactor generates a reacted cofactor.

7. The working electrode described in claim 6, wherein generated intermediary diffuses to a wired element and results in direct electron transfer from the first electrode reactive surface to the cofactor generating reacted cofactor, the reacted cofactor further enabling a reversible reaction of the intermediary back to the analyte.

8. The working electrode described in claim 4, wherein the enzyme is a dehydrogenase enzyme and the cofactor is an electron acceptor.

9. The working electrode described in claim 2, wherein a printing process is used to apply the first reactive chemistry to the first electrode reactive surface, the mediator within the first reactive chemistry being dispersed within a printable electrically conductive ink or paste.

10. An electrochemical sensor for measuring in-vivo analyte concentration within in subject, comprising:
a working electrode that includes
a working conductor having an electrode reactive surface;
a reactive chemistry being response to an analyte, the reactive chemistry including a mediator, an enzyme and a cofactor, the reactive chemistry being applied over the electrode reactive surface;
a pseudo-reference electrode that includes a combined counter-reference conductor; and
a transport material enables flux of the analyte to the reactive chemistry, the transport material being applied over the reactive chemistry and the pseudo-reference electrode.

11. The electrochemical sensor described in claim 10, wherein the pseudo-reference electrode further includes a counter-reference surface treatment.

12. The electrochemical sensor described in claim 11, wherein the mediator is a conductive polymer.

13. The electrochemical sensor described in claim 12, wherein the reactive chemistry is formed by electropolymerization of the mediator in the presence of the enzyme and the cofactor.

14. The electrochemical sensor described in claim 11, wherein a printing process is used to apply the reactive chemistry to the electrode reactive surface, the mediator within the reactive chemistry being a printable electrically conductive ink or paste.

15. The electrochemical sensor described in claim 13, wherein the reactive chemistry is defined by free elements of the enzyme and the cofactor entrapped within the polymerized mediator.

16. The electrochemical sensor described in claim 15, wherein the reactive chemistry is further defined by wired elements of enzyme and cofactor that are electrically connected to the working electrode and the mediator participates in transferring electrons.

17. The electrochemical sensor described in claim 16, wherein reaction of the analyte with free enzyme generates an intermediary and reaction of the free enzyme with the cofactor generates a reacted cofactor.

18. The electrochemical sensor described in claim 17, wherein generated intermediary diffuses to a wired element and results in direct electron transfer from the first electrode reactive surface to the cofactor generating reacted cofactor, the reacted cofactor further enabling a reversible reaction of the intermediary back to the analyte.

19. The electrochemical sensor described in claim 18, wherein the enzyme is a dehydrogenase enzyme and the cofactor is an electron acceptor.

20. The electrochemical sensor described in claim 10, wherein the pseudo-reference electrode is located on a side opposite the working electrode.
